(19) United States
(12) Patent Application Publication
(10) Pub. No.: US 2021/0307991 A1
(43) Pub. Date: Oct. 7, 2021

(54) SPINE LEVELING APPARATUS

(71) Applicant: Vincent Grover Kelley, Donalsonville, GA (US)

(72) Inventor: Vincent Grover Kelley, Donalsonville, GA (US)

(21) Appl. No.: 17/301,441

(22) Filed: Apr. 2, 2021

Related U.S. Application Data
(60) Provisional application No. 63/005,827, filed on Apr. 6, 2020.

(51) Int. Cl. A61G 13/12; A61B 50/24

(52) U.S. Cl. CPC A61G 13/1225; A61B 50/24; A61M 2210/1003; A61M 2205/586; A61G 2200/325

(57) ABSTRACT

The present invention provides a spine leveling apparatus comprising a rigid board which may be removably placed on a bed, surgical table or patient chair. The apparatus may further comprise two identical trays with articulating arms constructed and arranged for secure and removable securement on the rigid board. In use, the trays provide left and right working areas for the medical professional or caregiver. The apparatus further comprises a sheath which may be removably placed over the rigid board. The spine leveling apparatus may be beneficially used to position the patient in an optimal position for epidural or spinal placement. The trays further provide two clean and easily accessible working areas.

### CROSS-REFERENCE TO RELATED APPLICATION

This application claims the benefit of priority of U.S. Provisional Application No. 63/005,827 filed Apr. 6, 2020, the contents of which are herein incorporated by reference.

### BACKGROUND OF THE INVENTION

The present invention relates to medical devices, and more particularly, to a spine leveling device, and a spine leveling apparatus to assist in the placement of epidurals and spinals.

When placing an epidural or spinal for a woman in labor, or for pain patients, anesthesia providers, practitioners, and nurses may struggle with proper patient positioning due to factors including inconsistencies in the bed mattress, or the body habitus of a patient who is a pregnant woman or a patient with spine anomalies. Also it is particularly challenging to locate areas in patient rooms or small exam areas to easily access sterile epidural or spinal kits and other equipment such as sterile dressings, drugs and ultrasound machines.

Current options include rolling up some towels and placing them under the patient. This may be inconsistent and ultimately ineffective. As such, no effective options exist.

A need exists for a spine leveling device and a spine leveling apparatus to assist in the proper placement of epidurals and spinals. A further need exists for such an apparatus which is capable of use in multiple treatment settings.

### SUMMARY OF THE INVENTION

Advantageously, in one aspect, the present invention provides a solution which provides a device, an apparatus and a method for assisting in the placement of epidurals and spinals.

In one aspect, the present invention may comprise a spine leveling device comprising a rigid board which may be removably placed on a bed, a surgical table or a patient chair. The spine leveling device may be beneficially used to position a patient in an optimal position for epidural or spinal placement.

In another aspect, the present invention may provide a spine leveling device comprising a rigid board having a handle. The spine leveling device may be made of any suitable material, by any suitable fabrication process. In one embodiment the device may be made of rigid plastic.

In one aspect, the present invention may comprise a spine leveling apparatus comprising a rigid board which may be removably placed on a bed, a surgical table or a patient chair, and two identical trays with articulating arms constructed and arranged for secure and removable securement on the rigid board. In use, the trays provide left and right working areas for a medical professional or caregiver. The apparatus further comprises a sheath which may be removably placed over the rigid board.

The spine leveling apparatus may be beneficially used to position the patient in an optimal position for epidural or spinal placement, or any neuraxial procedures. The trays further provide two clean and easily accessible working areas. The spine leveling apparatus and its components may have any suitable dimensions, and may be made of any suitable materials, by any suitable fabrication process.

These and other features, aspects and advantages of the present invention will become better understood with reference to the following drawings, description and claims.

### BRIEF DESCRIPTION OF THE DRAWINGS

The preferred embodiment of the claimed subject matter will hereinafter be described in conjunction with the appended drawings provided to illustrate and not to limit the scope of the claimed subject matter, where like designations denote like elements, and in which:

FIG. 1 is a perspective view of the spine leveling apparatus in accordance with an exemplary embodiment of the present invention, shown in use;

FIG. 2 is a perspective view of the spine leveling apparatus; and

FIG. 3 is an exploded view of the spine leveling apparatus.

It is to be understood that like reference numerals refer to like parts throughout the several views of the drawings.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

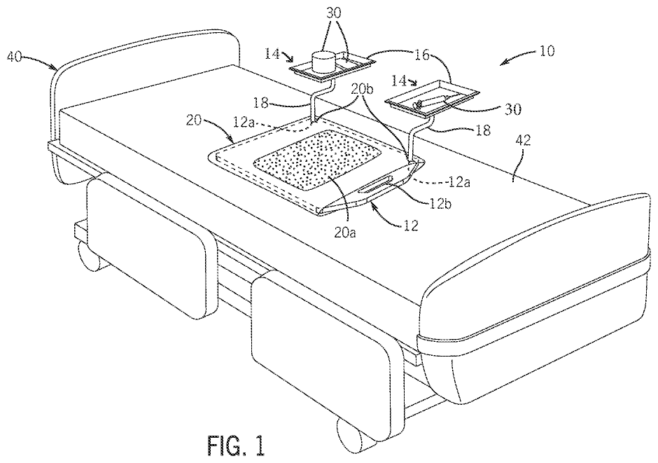

As used herein, “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, as there is no intention to be bound by any express or implied theory presented in the preceding technical field, background, brief summary or the following detailed description, it is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Broadly, one embodiment of the present invention provides a spine leveling apparatus and method for use. The present invention solves the need for a solution for effective placement of an epidural or spinal for a woman in labor or for pain patients, to assist anesthesia providers, practitioners and nurses with proper patient positioning. Also the present invention provides a solution for challenges associated with patient rooms and smaller examination areas, and provides easily accessible working areas for sterile epidural and spinal kits, and for other equipment such as sterile dressings, drugs and ultrasound machines.

The spine leveling apparatus of the present invention provides an unprecedented solution which may be used to position a patient in an optimal position for epidural or spinal placement, and also provides two clean and easily accessible work areas.

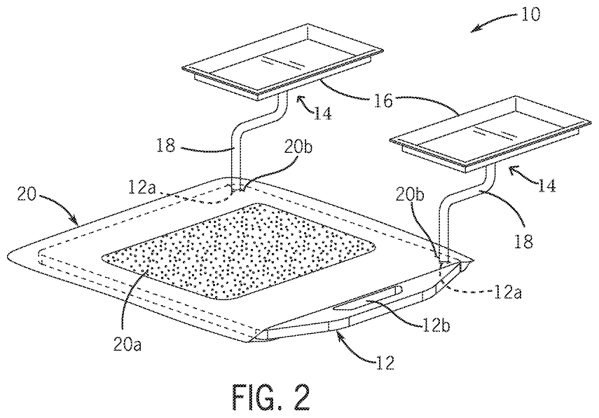

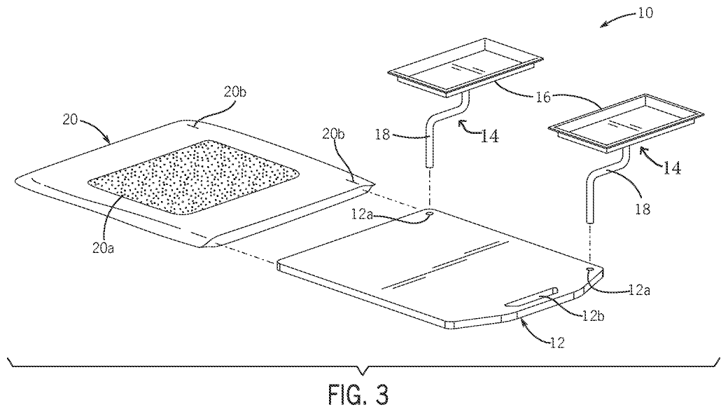

Referring now to the Figures, the spine leveling apparatus, shown generally at 10, is constructed and arranged for removable use in a surgical, hospital or home environment in which spine leveling is needed. FIG. 1 shows a perspective view of an exemplary embodiment of the present invention shown in use. FIG. 2 is a perspective view of the invention. FIG. 3 is an exploded view of the invention showing the elements or components in further detail.

As shown in the Figures, the present invention may provide a spine leveling device 12 comprising a rigid board having a handle 12b. The spine leveling device 12 may be made of any suitable material. In one embodiment the device may be made of rigid plastic.

As shown at FIGS. 1-3, the present invention may provide a spine leveling apparatus 10 which may comprise one or more of the following elements or components and combinations thereof.

a spine leveling device 12 comprising a rigid board;
a plurality of tray receptacles 12a of the spine leveling device 12;
a handle 12b of the spine leveling device 12;
a plurality of removable equipment tray assemblies 14;
a plurality of equipment trays 16;
a plurality of articulating support arms 18 for supporting the plurality of removable equipment trays 16;
a removable replaceable sheath 20 constructed and arranged to cover the spine leveling device 12;
an absorbent central portion 20a of the sheath 20; and
a plurality of slits 20b of the sheath 20 constructed and arranged to accommodate the plurality of articulating support arms 18.

As seen in the Figures, equipment 30 may be supported in the equipment trays 16 for use.

Also seen in the Figures, the spine leveling apparatus 10 may be used on a bed 40 with a mattress 42. The spine leveling apparatus may be used in any suitable setting, such as, for example without limitation, surgical, hospital, nursing home or care facility, medical office, or home care environments.

The spine leveling apparatus and its components may be made of any suitable material and made of any suitable fabrication method. Some components may be made of metal, stainless steel, plastic, silicone, carbon or combinations thereof. In an exemplary embodiment, the spine leveling device may be made of a rigid plastic.

In an exemplary embodiment the spine leveling apparatus may comprise a spine leveling device comprising a rigid plastic board. The rigid plastic board may be of any suitable shape or dimensions. In one embodiment, the rigid plastic board may have a generally rectangular shape, and may have a length which accommodates placement of the rigid plastic board upon a bed or patient table for a patient to sit upon. In some embodiments the rigid plastic board may have dimensions of 32 inches by 36 inches. In some embodiments, the rigid plastic board may further comprise a handle at a first end thereof.

The spine leveling apparatus may further comprise two identical trays with articulating arms. The rigid plastic board may comprise two receptacles to receive the articulating arms and support the two trays. It can also be seen that any suitable connecting element may be used to connect the articulating arms to the rigid plastic board. When the two trays are installed on the rigid plastic board, two working areas are provided, a right working area and left working area. The trays may be made of any suitable material, and may have any suitable dimensions. In some embodiments, the trays may have dimensions of about 11 inches by about 19 inches.

The apparatus may further comprise a thin plastic sheath constructed and arranged to removably cover the rigid plastic board. The plastic sheath may have an upper surface with a central absorbent area.

In an exemplary embodiment, the rigid plastic board may be precut to sit upon a standard hospital bed and provide the practitioner with superior patient ergonomics for spinal posture needed for easier epidural and spinal placement. The rigid plastic board presents a sharp contrast to lumpy or deflated air assisted mattresses.

The removable plastic sheath cover slides over the rigid plastic board with the open end thereof towards the foot of the bed. The removable plastic sheath upper surface central absorbent area may comprise a padded cotton center capable of absorbing liquids and body fluids, protecting both the board and bedsheets. The removable sheath may remain behind as the board is slid out easily via the open end of the removable sheath. The articulating arms of the two identical trays will fit into two holes in the plastic board that are on the proximal or near side of the provider doing the procedure. The custom plastic covers may include slits to accommodate the entry of the arms the trays going in easily. When combined, the apparatus and its components provide for better patient positioning and readily accessible work area.

In one embodiment, the present invention may provide a spine leveling device comprising a rigid board having a handle. The spine leveling device may be made of any suitable material. In one embodiment the device may be made of rigid plastic.

In an exemplary embodiment, the apparatus and its components may be made of any suitable material and fabricated by any suitable process. In some embodiments, a template would be made to make a reproducible mold for producing the spine leveling device comprising a rigid plastic board that is the base of the entire invention. The spine leveling device provides the spine stability by leveling the patient hips, which will in turn assist the practitioner with ease of placement of the epidural or spinal, hence less attempts and more patient comfort. The removable plastic sheath may be a plastic material with an upper portion including an upper surface with a cotton absorbent center. The removable plastic sheath may include one open end. The plastic sheath may be constructed and arranged to cover the spine leveling device, just long and wide enough to accommodate the rigid plastic board. The two trays may be designed to accommodate standard epidural and spinal equipment, and may be supported by articulating arms. The trays may be made of a plastic material. The articulating arms may be made of flexible plastic tubes constructed and arranged to be removably but securely mounted in the spine leveling device. The spine leveling device rigid plastic board is a vital component and the basis on which the invention is created. The removable, replaceable plastic covers are essential to keep the spine leveling device board clean for future uses. Although the trays are not essential, the trays make placement of spinals and epidurals even easier in that the treatment kits may be held in the trays and readily available at the side of the practitioner.

In an exemplary embodiment of the present invention, the provider would use this spine leveling apparatus to overcome a major pitfall in the placement of epidural or spinals, the poor hip and spine alignment associated with and caused by the current practices of performing epidurals and spinals on soft mattresses. The provider would place the spinal leveling device rigid board on the bed, covered in the plastic protective sheath and have the patient sit upon it. Placement of spinals and epidurals becomes easier, faster and more comfortable for the patient due to proper ergonomics. The two trays may be placed, if wanted, on each side for placing kits and supplies to have readily accessible and in a clean environment.

In an exemplary embodiment, the spinal leveling apparatus is designed for laboring women. In some embodiments, the spine leveling apparatus may be used in other procedures on the back, such as spinal taps in the Emergency Room, pain procedures and procedures performed on an operating room table. In some embodiments, the right and left adjustable trays provide a working surface for multiple medical procedures.

In summary, in exemplary embodiments, the present invention may provide a spine leveling device and a spine leveling apparatus.

The spine leveling device may comprise a flat planar board having an integral handle provided thereon. The spine leveling device may be made of any suitable material. In one embodiment, the spine leveling device may be made of rigid plastic material.

The present invention may further provide a spine leveling apparatus. The spine leveling apparatus may comprise a spine leveling device comprising a rigid flat planar board having a first end, a second end, a top surface and a bottom surface; and a removable replaceable sheath constructed and arranged to cover the spine leveling device, the sheath further comprising a top sheath portion, a bottom sheath portion, a first open end, a second closed end, and an absorbent top central portion.

In some embodiments, the spine leveling apparatus may provide a spine leveling device further comprising an integral handle at the first end thereof; and a plurality of tray receptacles located on the top surface thereof. The spine leveling apparatus may further comprise a plurality of equipment tray assemblies constructed and arranged for removable mounting in the plurality of tray receptacles of the spine leveling device. Each tray assembly may further comprise an equipment tray; and an articulating support arm. Each articulating support arm may have a first end connected to the equipment tray, and a second end constructed and arranged for removable insertion in a tray receptacle. The removable replaceable sheath may further comprise a plurality of sheath slits positioned on the top sheath portion to permit the plurality of articulating support arms to extend therethrough.

In some embodiments of the spine leveling apparatus, the plurality of tray receptacles may further comprise a first tray receptacle and a second tray receptacle; the plurality of tray assemblies may further comprise a first tray assembly and a second tray assembly; and the plurality of sheath slits may further comprise a first sheath slit and a second sheath slit.

For clarity, only those aspects of the system germane to the invention are described, and product details well known in the art are omitted. In addition, many embodiments of the present invention have application to a wide range of industries. To the extent the present application discloses an apparatus or a system, the method implemented by that apparatus or system is within the scope of the present invention. Further, to the extent the present application discloses a method, a system of apparatuses configured to implement the method are within the scope of the present invention.

It should be understood, of course, that the foregoing relates to exemplary embodiments of the invention and that modifications, variations and changes in detail may be made without departing from the spirit and scope of the invention as set forth in the following claims.

Page 10 of 10

What is claimed is:

1. A spine leveling device comprising a flat planar board having an integral handle provided thereon.

2. The spine leveling device of claim 1 made of rigid plastic material.

3. A spine leveling apparatus comprising:
a spine leveling device comprising a rigid flat planar board having a first end, a second end, a top surface and a bottom surface: and a removable replaceable sheath constructed and arranged to cover the spine leveling device, the sheath further comprising a top sheath portion, a bottom sheath portion, a first open end, a second closed end, and an absorbent top central portion.

4. The spine leveling apparatus of claim 3 wherein:
the spine leveling device further comprises:
an integral handle at the first end thereof; and
a plurality of tray receptacles located on the top surface thereof; and
the spine leveling apparatus further comprises:
a plurality of equipment tray assemblies constructed and arranged for removable mounting in the plurality of tray receptacles of the spine leveling device, each said tray assembly further comprising:
an equipment tray; and
an articulating support arm having a first end connected to the equipment tray, and a second end constructed and arranged for removable insertion in a tray receptacle; and
the removable replaceable sheath further comprises a plurality of sheath slits positioned on the top sheath portion to permit the plurality of articulating support arms to extend therethrough.

5. The spine leveling apparatus of claim 4 wherein:
the plurality of tray receptacles further comprise a first tray receptacle and a second tray receptacle;
the plurality of tray assemblies further comprises a first tray assembly and a second tray assembly; and
the plurality of sheath slits further comprise a first sheath slit and a second sheath slit.
